(19) United States
(12) Patent Application Publication
(10) Pub. No.: US 2010/0222855 A1
(43) Pub. Date: Sep. 2, 2010

(54) MEDICAL IMPLANTABLE LEAD WITH FLEXIBLE SEGMENTED SLEEVE (As Amended)

(76) Inventors: Rolf Hill, Jarfalla (SE); Olof Stegfeldt, Alta (SE); Armin Barlov, Jarfalla (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/599,621

(22) PCT Filed: May 15, 2007
(86) PCT No.: PCT/SE2007/000470

§ 371 Date: Nov. 10, 2009

(51) Int. Cl. A61N 1/00

(52) U.S. Cl. 607/116

(57) ABSTRACT

A medical implantable lead, which is adapted to be attached with a distal end to tissue inside a human or animal body, has a flexible sleeve in the distal end that protrudes a distance in an axial direction from the rest of the lead. When in a desired position, a force acting in an axial direction on the flexible sleeve will bring the sleeve to be compressed in the axial direction such that the surface area of the distal end will be enlarged. The flexible sleeve is formed with axial slots from an unbroken ring segment in an outermost distal edge of the sleeve and a distance in the proximal direction such that flexible segments are formed in the sleeve, wherein a force acting in an axial direction on the flexible sleeve will bring the sleeve to be compressed in the axial direction by folding outwards of the flexible segments.

### BACKGROUND OF THE INVENTION

1. Field of the Invention

The invention relates to a medical implantable lead, which is adapted to be attached with a distal end to tissue inside a human or animal body and that has a flexible sleeve in the distal end that protrudes a distance in an axial direction from the rest of the lead, of the type wherein, when in a desired position, a force acting in an axial direction on the flexible sleeve will bring the sleeve to be compressed in the axial direction such that the surface area of the distal end will be enlarged.

2. Description of the Prior Art

Medical implantable leads of various kinds and for various applications, e.g. for monitoring and controlling the heart in a human or animal body by means of a pacemaker, tend to become smaller and smaller in cross section. A medical implantable lead for pacemaker applications, for example, can have a diameter of less than 2 mm. This is advantageous in one aspect, since then the lead will be more flexible and take up less space. However, there are also risks with leads having a too small cross sectional dimension. In many cases the leads are namely adapted to be mounted to an organ inside the body, e.g. a heart wall, with its end surface abutting the organ and held by means of for example a helix, which is screwed into the organ. If the cross sectional dimension of the lead is too small in relation to its stiffness, it is a risk that the lead tip will perforate the organ during mounting of the lead and cause bleeding. This risk exists on the one hand when the distal end is pressed against the organ but before the lead is actually attached to it. In case the lead is provided with a rotatable helix for attaching to the organ, this risk also exists when the helix is screwed into the organ such that the distal end is drawn into the tissue by means of the rotating action of the helix. When the lead is attached to e.g. a heart, which performs large movements during function, there is also a risk that the heart wall will be perforated or injured during the course of a longer period of time when the lead is attached, due to abrasion or the like, if the lead is made with a too small cross sectional dimension in relation to its stiffness.

From United States Patent Application Publication No. 2007/0050003 A1, a medical implantable lead is known, which in one embodiment (FIGS. 7A-C) is provided with an elastic sleeve in a distal end which protrudes a distance beyond a distal end of the rest of the lead and which, when abutting the distal end against tissue or attaching the lead to the tissue by means of screwing a helix into the tissue, will buckle and expand to a diameter that is larger than the diameter of the distal end of the lead itself. However, there are several disadvantages with a medical implantable lead of this kind. To accomplish buckling of the sleeve, it is required a rather large force having to effect that, when the lead is mounted with its distal end in abutment against tissue, the sleeve will affect the lead with a force striving to push the lead from and disengage the helix from the tissue. This is not satisfactory since it may lead to that the helix might slip out of engagement with the tissue during long term usage due to movements in the tissue, e.g. a heart. To alleviate this effect, one possible solution would be to make the sleeve extremely elastic and flexible, but this would also have to effect that the contribution of the sleeve for preventing perforation into the tissue, would be reduced correspondingly. It would also have to effect that the risk for unintentional buckling of the sleeve, and hence an increased lead tip area, when introducing the lead through e.g. a narrow vein will increase, which might make it impossible to insert the lead.

### SUMMARY OF THE INVENTION

It is an object of the invention to provide an improved medical implantable lead by which the risk of perforation into tissue is eliminated or reduced and yet can ensure secure attachment to tissue.

The basis of the invention is the insight that the above object may be achieved by providing the medical implantable lead with means for varying the surface area of the distal end of the lead, such that the surface area has a minimum when introducing the lead into the body. Once the lead is localised inside the body, the surface area of the distal end can be enlarged when abutting the distal end against an organ or other tissue.

According to the invention, the enlargement of the surface area is accomplished by mechanically folding material portions at the distal end. More precisely, the distal end of the lead has a flexible sleeve, of e.g. silicone plastics, which protrudes a distance beyond the rest of the medical implantable lead. The outermost, distal end of the flexible sleeve is unbroken and hold together as a ring segment, whereas it is provided with slots in the axial direction of the lead, from the unbroken ring segment and a distance in the proximal direction, such that flexible segments are formed in the sleeve. When applying an axial force on the sleeve in the axial direction towards the proximal end, the flexible segments will be folded outwards and increases in this way the surface area of the lead end. The axial force can be applied in different ways, e.g. by pressing the distal end against tissue or by letting a rotatable helix engage with some kind of engagement formation on the ring segment, such as an engagement post on the inside of the ring segment or an aperture in a disk positioned over the distal end of the ring segment. The latter requires, as in the embodiment described and illustrated below, that the helix is rotatable from the proximal end of the lead, by any suitable means known in the art, but is not displaceable in the axial direction of the lead during rotation. The displaceable arrangement of the helix is normally done to withdraw the helix into a tubular header such that the helix is accommodated in the header to prevent it from penetrating and causing damage during introduction into the body. In this case this is not necessary since the flexible sleeve will cover the helix during introduction.

Normally, at least four flexible segments are required for performing the folding of the flexible segments without having to use an excessive force to accomplish the folding of the sleeve. The length of the slotted portion of the sleeve is preferably from the ring segment to the position of the distal end of the rest of the lead and can suitably have a length of about 1,5-2 mm.

To facilitate folding of the sleeve segments, they can, as in a hereinafter described and illustrated embodiment, be provided with one or more grooves around the circumference of the sleeve on the side of the sleeve towards which the sleeve segments will be folded. More precisely, on the inside of the sleeve in the middle of each sleeve segment and preferably also on the outside in the respective ends of the segments, wherein the innermost groove is located at the position of the distal end of the rest of the lead.

It is preferred for the sleeve in the folded and mounted state, not to press outwardly in the axial direction with a force against the tissue, which might lead to that the attachment to the organ in the course of time might become disconnected. This may be achieved in different ways, e.g. by a helix being in engagement with an engagement formation on the ring segment, such as a post on the inside of the unbroken ring or an aperture in a disk, or by means of a circumferential groove, which does not divide each segment into two equal parts but one part is longer than the other such that a dead centre locking is achieved of the flexible segments in the folded position. It is also possible to make the sleeve in a preformed folded position and let the helix stretch out the sleeve during insertion by means of engagement with an engagement formation. When attaching the lead to tissue by rotating the helix, the sleeve will be folded to the preformed shape.

### BRIEF DESCRIPTION OF THE DRAWINGS

FIG. 1 is a perspective view of the distal end of a medical implantable lead in accordance with the invention, in an introducing state.

FIG. 2 is a perspective view of the lead of FIG. 1 in a partly compressed state, when the sleeve segments are being folded outwardly.

FIG. 3 is a perspective view of the lead according to FIGS. 2 and 3 in a mounting state, with the flexible segments folded outwardly.

FIG. 4 is a longitudinal section through the distal end of the medical implantable lead in accordance with the invention, in the introducing state.

FIG. 5 is a longitudinal section through the distal end of the medical implantable lead in accordance with the invention being introduced through a vein.

FIG. 6 is a longitudinal section according to FIG. 5, when the lead is being attached to a heart wall and the flexible segments are folded outwardly.

FIG. 7 is a longitudinal section of the medical implantable lead in accordance with the present invention, in a mounted state.

FIG. 8 is a longitudinal section through the medical implantable lead in accordance with the invention in an extracting state, when being extracted from a patient through a vein and with the sleeve segments being forwardly folded.

### DESCRIPTION OF THE PREFERRED EMBODIMENTS

In the drawings is illustrated one embodiment of the invention in form of a medical implantable lead adapted to be used in connection with a pacemaker or with an implantable cardioverter defibrillator. Accordingly, the lead comprises a considerably long electrical lead 1, of which only a distal end is shown in the drawings. A pacemaker is adapted to be connected to a proximal end of the lead, whereas a distal end 2 is adapted to be attached to a heart wall 3. As is best seen in FIGS. 4-8, the lead is, in a distal end, provided with a rigid tubular header 4 of metal or plastics, in the end of which a helix 5 is rotatably arranged. The helix can be rotated from the proximal end of the lead, by suitable means known in the art but not shown in the drawings, such as a stylet or a coil. On the outside of the header 4 the lead is provided with a sleeve 6 of a resilient material, which is extended a distance beyond the distal end of the header and covers the helix 5 completely in an introducing state, as is shown in FIGS. 1, 4 and 5.

The sleeve is formed with an unbroken ring segment 7 in its distal end and axial slots 8, which extends from the unbroken ring segment 7 and a distance inwards, preferably to the distal end of the rest of the lead. In this way flexible segments 9 are formed between adjacent slots 8. On the inner side, the unbroken ring segment 7 is provided with an engagement means in form of a post 10, which is in engagement with the helix winding 5.

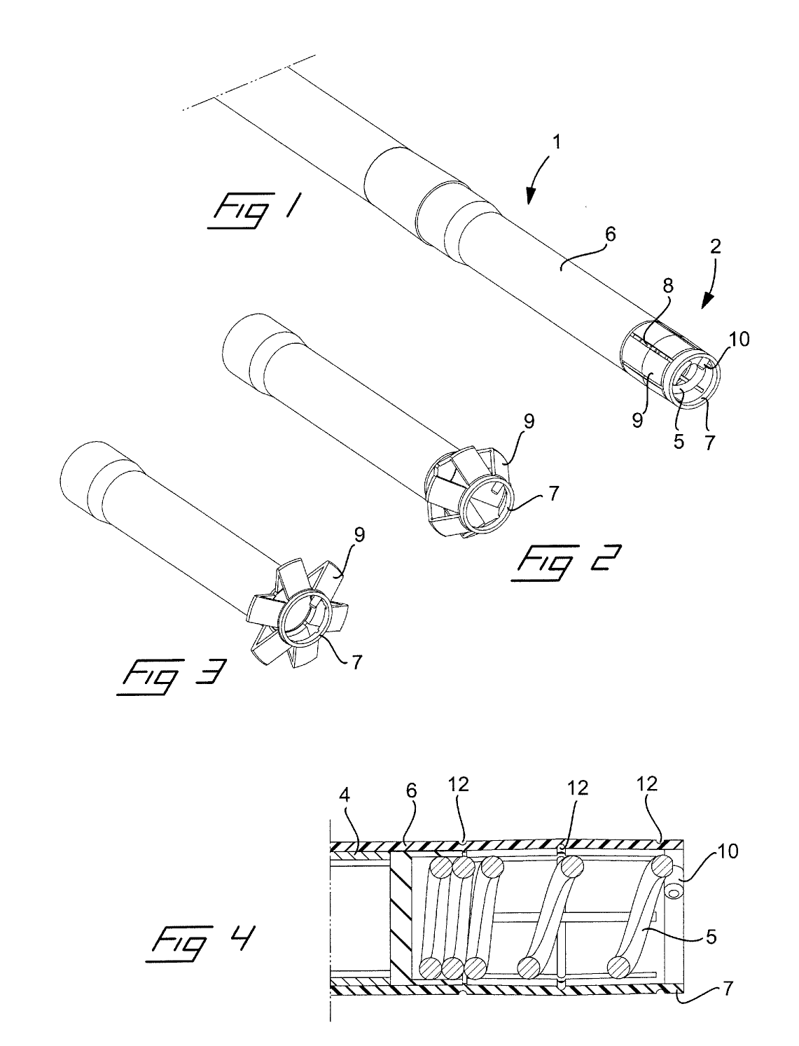

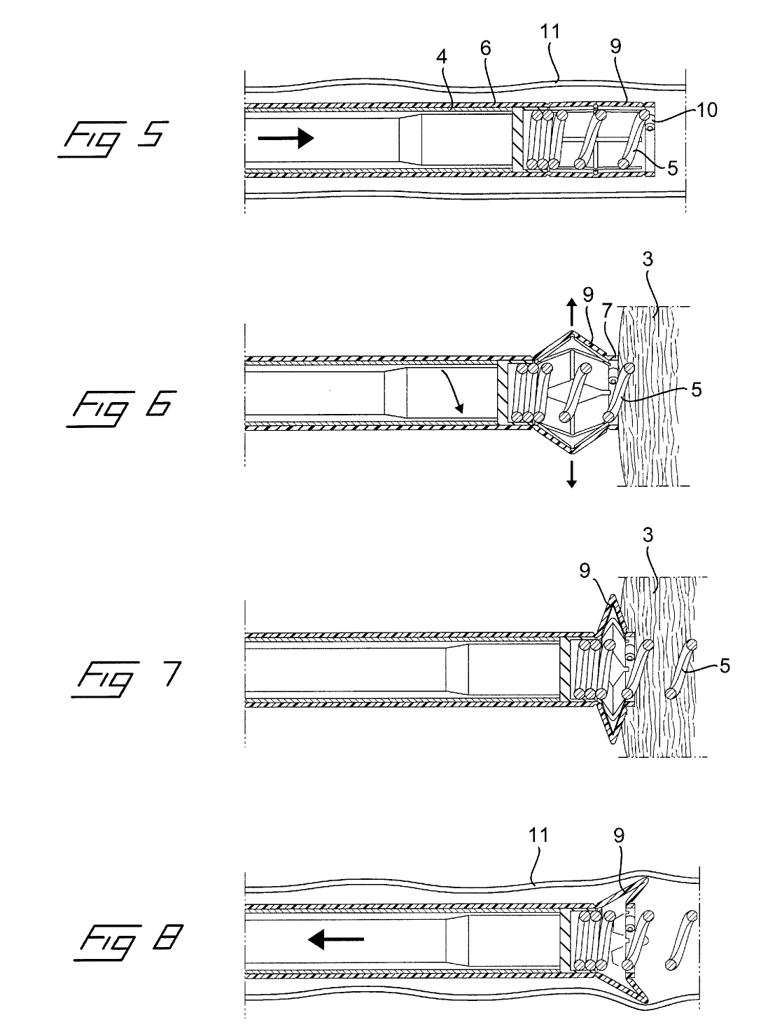

When implanting the lead into a human or animal body, e.g. by introducing it through a vein 11, as is illustrated in FIG. 5, the lead is in the introducing state as is also shown in FIG. 1. In this state the sleeve 6 covers the helix 5 and prevents it from penetration into tissue. When the lead has been introduced into the body and the distal end of the lead is bearing against tissue 3, at a location to be mounted in, the helix is caused to rotate by means of a rotary transmitting structure, such as a stylet or a coil, by means of which a rotary movement can be transmitted from a proximal end to the distal end of the lead. When rotating the helix 5, its tip will penetrate into the tissue 3 at the same time as the unbroken ring segment 7 will be displaced towards the header 4 due to the engagement of the post 10 with the helix winding 5, as is illustrated in FIG. 6. This will bring the flexible segments 9 to fold outwards and when the unbroken ring segment 7 has reached its inner end position, as is illustrated in FIG. 7, the flexible segments 9 will be folded and oriented in a substantially radial direction around the periphery of the lead such that the surface area of the distal end of the lead is considerable increased in relation to the mounting state.

To facilitate folding of the flexible segments, these are provided with grooves 12 in a circumferential direction of the lead in the areas where the segments 9 are folded, i.e. in each end of the segments 9 as well as in the middle of each segment. The grooves 12 are best seen in FIG. 4 and, as can be seen, the grooves are formed on the side towards which it is to be folded, i.e. on the outside at the ends of the segments and on the inside in the middle of the segments.

In FIG. 8 is illustrated a situation in which the lead are to be retracted from inside the body. This situation may occur several years after implanting into the body and by that time, the distal end can be more or less overgrown by tissue such that folding back of the sleeve 6 to the introducing state in FIG. 1 may be difficult or even impossible. However, due to the highly flexible characteristics of the sleeve, the flexible segments can be “double folded”, as is illustrated in FIG. 4, when the lead is being extracted and met with resistance from e.g. the inner walls of a vein 11. In this way the extraction will be facilitated.

Although modifications and changes may be suggested by those skilled in the art, it is the intention of the inventors to embody within the patent warranted heron all changes and modifications as reasonably and properly come within the scope of their contribution to the art.

We claim as our invention:

1-9. (canceled)

10. A medical implantable lead comprising:
a lead body adapted for in vivo implantation in a living subject, said lead body having a distal end configured for in vivo attachment to tissue in the living body;
said distal end comprising a flexible sleeve that protrudes a distance in an axial direction from a remainder of said lead body;
said flexible sleeve comprising a plurality of axial slots extending in said axial direction and proceeding from an unbroken ring segment of said sleeve at an outermost distal edge of said sleeve, said axial slots forming a plurality of flexible segments in said sleeve; and
said flexible segments causing said sleeve, when a force is applied thereto in said axial direction, to compress in the axial direction by the segments folding outwardly from said sleeve and from said lead body.

11. A medical implantable lead as claimed in claim 10 wherein each flexible segment comprises a circumferential groove on an interior side of the segment, forming a hinge that facilitates folding of the segment.

12. A medical implantable lead as claimed in claim 11 wherein each flexible segment comprises a further circumferential groove on an exterior side of the segment, also forming said hinge.

13. A medical implantable lead as claimed in claim 12 wherein, in each segment, an innermost circumferential groove is positioned at the distal end of said remainder of said lead.

14. A medical implantable lead as claimed in claim 10 comprising a rotatable helix in said distal configured for engagement with said tissue by screwing said helix into the tissue, and wherein said sleeve comprises an engagement structure on a distal ring segment that engages said helix as said helix is screwed into the tissue, forming said force applicator that compresses said sleeve.

15. A medical implantable lead as claimed in claim 14 wherein said engagement structure is a post on an interior of said ring segment.

16. A medical implantable lead as claimed in claim 15 wherein said engagement structure is an aperture in a disk on said ring segment.

17. A medical implantable lead as claimed in claim 10 wherein said flexible sleeve comprises at least four of said segments.

18. A medical implantable lead as claimed in claim 10 wherein said segments of said sleeve are also movable, after being folded outwardly, to an extracting state by applying a force to said sleeve that bends said segments in a distal direction to reduce said surface area of said distal end.
